(19) United States
(12) Patent Application Publication
(10) Pub. No.: US 2012/0059451 A1
(43) Pub. Date: Mar. 8, 2012

(54) Method of Manufacturing a Polymeric Stent Having Reduced Recoil

(76) Inventors: Qiang Zhang, Annandale, NJ (US); Joseph H. Contiliano, Stewartsville, NJ (US)

(21) Appl. No.: 13/226,850

(22) Filed: Sep. 7, 2011

Related U.S. Application Data
(60) Provisional application No. 61/380,857, filed on Sep. 8, 2010.

(51) Int. Cl. A61F 2/82; B29C 35/08; D01D 5/24

(52) U.S. Cl. 623/1.15; 264/171.26; 264/400

(57) ABSTRACT

Methods of manufacturing polymeric intraluminal stents, and stents made by such methods, are disclosed. The methods provide for manufacturing polymeric intraluminal stents by inducing molecular orientation in the stents by radial compression thereby providing stents with low recoil post-deployment.

### FIELD OF THE INVENTION

The present invention relates to a method of manufacturing polymeric intraluminal stents, such as balloon expandable or partially balloon expandable stents, and more particularly to polymeric intraluminal stents that have reduced recoil.

### BACKGROUND OF THE INVENTION

Intraluminal stents are generally cylindrically shaped medical devices implanted within a body lumen having an initial reduced diameter and deployed at the desired location within the lumen by radially expanding the stent to a second larger diameter, typically using a balloon catheter. Stents are typically used by the medical professional to increase the patency of a lumen or body structure, often in vascular system applications. A stent should possess various requisite qualities and characteristics including a certain degree of flexibility in order to be readily maneuvered through tortuous vascular pathways, and in order to conform to nonlinear vessel walls when deployed and expanded. When expanded, an intraluminal stent should exhibit certain mechanical characteristics, including the ability to maintain vessel patency by providing an acute and/or chronic outward force that will help to remodel the vessel to its intended luminal diameter, prevent excessive radial recoil upon deployment and have sufficient ductility so as to provide adequate coverage over the full range of desired and intended expansion diameters. After deployment and expansion, an intraluminal stent acts as a support structure by providing an outwardly directed radial force to the vessel walls to maintain patency of the lumen.

Stents for balloon expandable applications are typically manufactured from a material having sufficient elongation at break to allow the stent to be crimped in a low profile state for insertion into the vasculature or other body lumen, while also enabling the stent to withstand the excessive strains experienced during balloon expansion without damage. Metal alloys such as 316L stainless steel and L605 CoCr that are currently utilized to manufacture balloon expandable stents typically possess an elongation at break of approximately forty percent, thus allowing stents manufactured from such materials to deploy and expand in response to forces applied by a pressurized balloon without breaking. Typical non-elastomeric implantable bioabsorbable polymers such as PLA (polylactic acid), PGA (polyglycolic acid), and copolymers of PLA and PGA (PLGA) have relatively low elongation at break values, typically less than fifteen percent. In addition, the tensile strength and tensile modulus of these polymers are orders of magnitude less than the metals previously mentioned. It is highly desirable to have a material with improved elongation at break, i.e., ultimate strain capacity, without compromise to the modulus or ultimate strength of the material necessary in order to provide a stent with sufficiently high radial strength while having minimal stent recoil. Manufacturing methods have been developed to increase elongation at break while maintaining or improving material strength and stiffness, allowing the stent wall thickness to be kept small, thereby resulting in better device flexibility and less resistance to impede blood or other bodily fluid flow.

Polymer chain orientation through mechanical deformation is a known way to induce added toughness in polymer-based materials. One method to enhance the mechanical properties of polymeric stents is to induce polymer orientation in a polymer tube or sheet that is used to form the stent. This can be done by applying mechanical forces in various directions in the desired direction of orientation (for example, axially, radially, or both (biaxially)). It is known in this art to utilize methods of orienting polymeric tubing for use as stents. It is well known in the art that molecular orientation, or the induction of polymer chain alignment, can enhance the material properties such as strength and toughness. Strength of material is typically defined to mean the amount of force the material can withstand prior to failure. Material toughness is typically defined to mean the amount of energy the material can absorb prior to failure. Molecular orientation can be achieved by heating the material above the glass transition temperature (Tg) of the material, while applying a force or forces to the material to provide the desired polymer orientation, and then cooling the material to below the Tg.

Various methods are disclosed in the art of using axial, radial, and biaxial oriented tubing to manufacture polymeric stents having enhanced material properties, in which the molecular orientation is induced in the polymer while in some intermediary form (e.g., tube, sheet, etc.), prior to being formed into a stent (e.g., machining, rolling or laser cutting, etc.). Stents made using methods known in the art are typically made from oriented tubing with a smaller outer diameter (OD) than the OD of the expanded stent after balloon deployment in the body. The OD of the stent is typically manufactured at a size between the desired small crimped size needed to enable suitable delivery and final deployment size and final desired deployed size. For example, it is known to utilize methods of using tubing produced via various processes, including melt processing and solvent casting processes, orienting the tubing in various ways to affect and enhance material properties, and then creating stents from the treated tubing. Although polymer orientation in one direction can enhance material properties in that direction, there is potential to compromise the material properties in an orthogonal direction to the orientation direction. By orienting the tubing prior to cutting the stent, the molecular orientation and hence the enhancement of material properties is created along the axes (typically longitudinal and/or circumferential) of the tubing used to create the stent, but not necessarily in the appropriate directions as dictated by the specific stent strut configuration or geometry for optimal performance after deployment.

All of the above-mentioned methods provide polymeric stents having molecular orientation, and when such stents are then expanded to a larger diameter size (e.g., after deployment), they are at risk of experiencing stent recoil. Stent recoil is conventionally defined as a percentage drop in stent cross-sectional diameter over time. It can be due to having a stent radial stiffness insufficient to withstand compressive vessel forces, as well as inherent material relaxation in polymers such as creep. Material relaxation in polymers may occur because the induced orientation in the polymer is not necessarily in equilibrium, and thus there exists an inherent driving force in the polymer to eventually revert back to its pre-oriented state. In addition, the amorphous regions of the polymer structure may undergo densification, which can lead to material brittleness. Additional thermal methods are known which attempt to mitigate the effects of aging process of polymers, in particular applications directed toward stents constructed from oriented polymer tubing, in order to prevent or mitigate adverse effects on stability and shelf life over time. Thermal techniques to combat polymer aging in oriented polymers are challenging to implement and typically rely on induced crystallinity for polymer stability. Some bioabsorbable polymers, co-polymers, or blends thereof do not readily crystallize, and an associated disadvantage is that an increase in crystallinity in bioabsorbable polymers may often be linked with increased absorption times, a phenomenon that is not entirely desirable. Furthermore, it is known in the art that crystalline regions in a semicrystalline absorbable polymer have a greater tendency to elicit a less benign tissue response in the body compared to amorphous polymeric materials. It is generally known that inducing crystallinity while preserving material toughness is a challenge, and it is also known that certain materials lack the ability to crystallize, so the availability of suitable bioabsorbable polymeric materials is severely limited.

Polymeric stents are known that are expanded radially outward through the facilitation of heat applied to the stent to raise the temperature of the stent to above the Tg of the material thus inducing molecular orientation in the stent in situ, and in some embodiments, the polymer of the stent may have a Tg at or below body temperature. Several examples of polymer blend systems useful in such stents, such as those containing trimethylene carbonate or poly(epsilon-caprolactone), which contain a lower Tg are described in the art. These compositions typically result in a stent material with lower modulus and strength, and can exacerbate recoil in a deployed stent when used in the body above their Tg. Additionally, heating a stent to effect deployment is not desirable since it requires that an additional step be added to the surgical procedure, may introduce procedural variabilities between surgeons, and can possibly cause thermal damage to body tissues.

Other art discloses polymer orientation methods performed to a stent itself rather than orienting the polymer tubing or sheet which is used to construct the stent. For example, the idea of orienting a stent in situ with the addition of heat through a heated catheter has been disclosed. It is believed that this method is disadvantageous since the amount of orientation induced in this manner can vary depending on surgeon technique, and, as previously mentioned, the introduction of heat to deploy a stent in the body is not desirable and may cause tissue or cell damage.

It is known, for example, to reduce stent recoil in a polymeric stent by plastically deforming tubes to a larger size diameter and then annealing them to shrink the diameter to an intermediate size. Subsequent to balloon deployment from this intermediate size, the stents are claimed to have lower recoil than if deployed from the starting size. This method does not seek to orient the stent directly, furthermore, plastically deforming the stent at a relatively low temperature may predispose the stent to cracking, and there are limited materials that can withstand this plastic deformation prior to any thermal treatment.

It is also known to use a method whereby polymeric cylindrical devices (stents) are first heat treated at an elevated temperature to “educate” the stent to remember a predetermined shape and diameter. Stents are then mounted on balloon catheters and subjected to a milder heat/temperature crimp cycle with a temperature at or slightly above Tg; a temperature sufficiently high to allow deformation of the device but not high enough to allow the chains to reorganize and erase memory of the final shape of the educated device. Education times and temperatures need to be discerned for a particular material used and may depend upon the material's ability to form crystallites. In addition, the crimping step is restricted to occur at temperatures at or only slightly above the Tg so as to not interfere with the prior-induced education thermal history.

Another known method provides for stents of larger size diameters that are thermally educated at a first higher temperature and then crimped at a second temperature below the stent material's glass transition temperature down to a suitable diameter equal to the insertion size. Lower recoil is claimed since the tube has been trained to go back to its “educated” size. A challenge associated with this method is maintaining the stent in the crimped configuration. This method is distinctive and different in that the crimping step is expressly required to occur below the Tg of the material, so as not to interfere with the “educated” shape that was induced in the prior thermal step.

Also known in this art is a method of orienting a stent prior to insertion in the body (versus orienting the tubing prior to constructing the stent) to induce molecular orientation in regions of the stent strut architecture. The process includes orienting stents from a small size to a larger interim size, wherein the diameter of the balloon deployed stent in the body is at an even larger size.

Accordingly, there is a need in this art for novel polymer-based stents and a novel manufacturing process that overcome the disadvantages that may be associated with currently known and available polymeric stents and manufacturing processes.

Therefore, it is an object of the present invention to provide novel processes for manufacturing intraluminal polymeric stents applicable to polymeric, and more specifically absorbable polymeric, materials which are naturally less tough and more brittle than currently used metal alloys.

It is a further object of the present invention to provide novel processes for manufacturing polymeric intraluminal stents that result in stents having low recoil or diameter contraction after implantation.

It is yet a further object of the present invention to provide processes for manufacturing intraluminal polymer stents that do not require the stents to be educated by subjecting the stents to temperature sufficiently high enough above the Tg for prolonged lengths of time.

A further object of the present invention is to provide novel processes for manufacturing polymeric intraluminal stents, wherein the stents produced by the processes have low recoil without the need to apply heat to the stent that is higher than body temperature to effect stent deployment, thereby not requiring an extra heating procedure or change to the current traditional methods of stent/balloon catheter deployment.

Still yet a further objective of the present invention is to provide novel processes for manufacturing intraluminal polymer-based stents that produce stents that have low recoil and are compatible with both amorphous and partially crystalline polymers without relying on the capacity of the material to crystallize or the level of crystallinity to maintain material stability.

Another object of the present invention is to provide novel processes for manufacturing intraluminal polymer-based stents resulting in stents that have low recoil while also being compatible with amorphous materials; amorphous regions of absorbable materials generally have a more benign tissue response compared to crystalline regions.

An additional object of the present invention is to provide novel processes for manufacturing intraluminal polymer based stents resulting in novel stents that have low recoil using bioabsorbable polymers that have faster absorption rates than highly crystalline PLA and other bioabsorbable materials that may remain in the body for 24 to 36 months.

Still yet another object of the present invention is to provide novel processes for manufacturing intraluminal polymer-based stents resulting in stents that have low recoil, while also being compatible with bioabsorbable polymers that have glass transition temperatures both above and below 60° C.

### SUMMARY OF THE INVENTION

A novel method of manufacturing a polymeric stent is disclosed. Initially, a polymeric stent is formed from a polymeric material. The stent has a first inner diameter and a first outer diameter, and the stent has a plurality of openings forming struts, wherein the first inner and first outer diameters of the stent are substantially equal to the inner and outer diameters of the stent post-deployment. The stent is then heated to a temperature sufficiently above the Tg of the material. The stent is then radially compressed at the temperature such that it has a reduced second inner diameter and a reduced second outer diameter, wherein the second inner and outer diameters are smaller than the first inner and outer diameters, respectively. The stent is cooled in the compressed configuration. The treated stent has substantially no recoil after deployment.

Another aspect of the present invention is a novel polymeric stent manufactured using the novel process of the present invention. Initially, a polymeric stent is formed from a polymeric material. The stent has a first inner diameter and a first outer diameter, and the stent has a plurality of openings forming struts, wherein the first inner and first outer diameter of the stent are substantially equal to the inner and outer diameters of the stent post-deployment. The stent is heated to a temperature sufficiently above the Tg of the material. The stent is then radially compressed at the temperature such that it has a reduced second inner diameter and a reduced second outer diameter, wherein the second inner and outer diameters are smaller than the first inner and outer diameters, respectively. The stent is cooled in the compressed configuration. The stent produced by this process, when expanded to a size substantially equal to the first inner diameter and the first outer diameter, has substantially no recoil.

Yet another aspect of the present invention is a surgical procedure to open a vessel lumen by deploying and expanding a novel stent of the present invention in the vessel lumen.

The foregoing and other features and advantages of the present invention will become more apparent from the following description and accompanying drawings.

### BRIEF DESCRIPTION OF THE DRAWINGS

FIG. 1 is a two-dimensional representation of stent in laser cut condition (pre-orientation) used in Example 1; the stent design contains 18 strut columns.

FIG. 2 is a two-dimensional representation of stent in laser cut condition (pre-orientation) used in Example 2; the stent design contains 14 strut columns.

FIG. 3 is a two-dimensional representation of stent in laser cut condition (pre-orientation) used in Examples 2 and 3; the stent design contains 15 strut columns.

FIG. 4 is a photograph of a stent made in accordance with Example 2 in the manufactured (deployed) size.

FIG. 5 is a photograph of the stent of FIG. 4 after radial compression orientation to a size appropriate for stent delivery.

FIG. 6 is a schematic diagram illustrating a cross-sectional view of a tubular stent shown manufactured to desired deployment size A; the stent following radial compression and orientation to a smaller diameter B; and, the stent after deployment with a balloon to final deployment diameter C.

### DETAILED DESCRIPTION OF THE INVENTION

The novel polymeric stents of the present invention utilize polymer orientation applied to polymeric stents prior to stent implantation, in a way that any polymeric material relaxation which occurs will tend to increase (not decrease) stent cross-sectional size (thus limiting effects on stent recoil) in contrast to decreasing stent size and contributing to stent recoil. The novel methods and stents of the present invention may utilize a wide range of polymeric materials with more desirable absorption rates and there is not a requirement for the ability of the material to crystallize to combat material relaxation. The methods of the present invention may be used with balloon expandable stents, and may also be used with other expanding means and devices.

The novel methods and processes of the present invention are directed to substantially tubular intraluminal polymer-based medical devices having a longitudinal axis and a radial axis of various (but not limited to) stent strut architectures, including conventional architectures known in the art. The biocompatible materials for implantable medical devices of the present invention may be utilized for any number of medical applications, including vessel patency devices such as vascular stents, biliary stents, renal stents, pancreatic duct stents, fallopian tube stents, ureter stents, sinuplasty stents, airway stents, vessel occlusion devices such as atrial septal and ventricular septal occluders, patent foramen ovale occluders and orthopedic devices such as fixation devices.

The terms ID and OD as used herein are defined to have their conventional meanings of inner diameter and outer diameter, respectively.

The polymeric tubes used to manufacture the stents of the current invention may be prepared from various conventional processes, including melt and solution. Typical melt processes include injection molding, extrusion, fiber spinning, compression molding, blow molding, etc. Typical solution processes include solvent cast tubes and films, electrostatic fiber spinning, dry and wet spinning, hollow fiber and membrane spinning, spinning disk, etc. Pure polymers, blends, and composites can be used to prepare the stents. The precursor material can be a tube or a film that is prepared by any of the processes described above.

The novel process of the present invention involves first creating a stent by cutting a tubular member (through any conventionally known means in the art) into a stent having an expandable structure, wherein the tube has a diameter equal to the final expanded or near final expanded size and configuration desired. Stents of the present invention are constructed from polymeric tubing of length, diameter, and wall thickness substantially similar to the desired dimensions after the stent would be balloon-deployed. In a similar manner, the stents of the present invention can be made from tubes that are made using a process wherein the tube is made by rolling a sheet of polymeric material into a polymeric tube, and then cutting or machining the tube to form a stent. The tubing size can be made substantially equal or in some cases larger than the desired final diameter of the device when an increased outward residual force against the vessel is desired. The tubing diameter can be substantially equal to or in some cases slightly greater than the desired diameter after the stent to a prescribed degree. The stent can be manufactured from tubing through any known processes such as laser cutting, other micromachining, photoetching, etc. The stents of the present invention may also be made by other conventional methods, including, for example, injection molding and casting.

The terms polymer-based or polymeric are used interchangeably herein and refer to stents made from biocompatible, bioabsorbable or nonabsorbable polymers, or stents which are composites utilizing a polymer matrix with other biocompatible filler materials (ceramic or metal).

After a bioabsorbable polymeric stent has been deployed and expanded in the lumen of a vessel in vivo, the body responds by encasing the stent walls within the wall of the vessel in the natural healing process. The stent will then subsequently absorb and/or degrade in the body over time to minimize the likelihood of embolization of any breakdown fragments of the stent. Unlike metal stents, bioabsorbable stents offer a potential advantage in that repeat stenting within the same vessel location may be possible. A bioabsorbable stent may also allow vessels to positively remodel over time with an eventual return of natural flexibility and vasomotion.

The present invention provides novel processes for making polymer-based stents and novel stents manufactured from said processes, wherein the stents over time fully recover any acute recoil that may occur during initial stent deployment Polymer-based materials encompass both bioabsorbable and nonabsorbable biocompatible polymers, as well as polymer-based composite materials wherein one or more biocompatible ceramic or metallic additives can be added to the polymer-based material to provide certain material properties such as modulus or radiopacity. The bioabsorbable polymers used in the processes and stents of the present invention may encompass polymers that are either bulk eroding or surface eroding in nature. The devices herein described may be used in conjunction with pharmaceutical agents (such as known anti-restenotic and/or anti-thrombotic agents for example), cells, bioactives, radiopaque markers, as is currently known in the stent literature. The present invention may also be used in conjunction with various known thermal treatments discussed in the art (such as stress relieving or annealing) to reduce stress or create crystallization within the device if desired. The novel stents of the present invention include, but are not limited to, both balloon expandable and partially balloon expandable stents.

It is recognized that the term “stent” of the present invention could be any tubular polymeric construct implanted into a variety of body lumens to serve either a scaffolding or drug delivery function such as, but not limited to renal, urethral, coronary, carotid, biliary, pancreatic duct, gut, fallopian tubes, peripheral stents, etc., typically expanded from a smaller diameter to a larger diameter when placed in the body. The novel methods of the present invention produce novel polymeric stents that have improved capacity to maintain their larger diameter size (reduced stent recoil) over time after being implanted in the body. It is also recognized that balloon expandable stents refer to tubular polymer constructs that are deployed within the body by plastically deforming the material by inflating and deflating a balloon catheter, and that other equivalent means of plastically deforming the tubular constructs to appropriate deployment sizes can be utilized.

The polymer tubing may be prepared from polymeric materials such as biocompatible, bioabsorbable or nonabsorbable polymers. The selection of the polymeric material used to prepare the polymeric tubing according to the invention is selected according to many factors including, for example, the desired absorption times and physical properties of the materials, and the geometry of the intraluminal stent. Examples of nonabsorbable polymers include polyolefins, polyamides, polyesters, fluoropolymers, and acrylics. Biocompatible, bioabsorbable and/or biodegradable polymers consist of bulk and surface erodable materials. Surface erosion polymers are typically hydrophobic with water labile linkages. Hydrolysis tends to occur fast on the surface of such surface erosion polymers with no water penetration in bulk. The initial strength of such surface erosion polymers tends to be low however, and often such surface erosion polymers are not readily available commercially. Nevertheless, examples of surface erosion polymers include polyanhydrides, such as poly (carboxyphenoxy hexane-sebacic acid), poly(fumaric acid-sebacic acid), poly(carboxyphenoxy hexane-sebacic acid), poly(imide-sebacic acid) (for example, in a mole ratio of 50/50), poly(imide-carboxyphenoxy hexane) (for example, in a mole ratio of 33/67), and polyorthoesters (i.e. diketene acetal based polymers).

Bulk erosion polymers, on the other hand, are typically hydrophilic with water labile linkages. Hydrolysis of bulk erosion polymers tends to occur at more uniform rates across the polymer matrix of the stent. Bulk erosion polymers exhibit superior initial strength and are readily available commercially. Examples of bulk erosion polymers include poly (alpha-hydroxy esters) such as poly (lactic acid), poly (glycolic acid), poly (caprolactone), poly (p-dioxanone), poly (trimethylene carbonate), poly (oxaesters), poly (oxaamides), and their co-polymers and blends. Some commercially readily available bulk erosion polymers and their commonly associated medical applications include poly (dioxanone) [sutures are sold under the tradename PDS available from Ethicon, Inc., Somerville, N.J.], poly (glycolide) [sutures are sold under the tradename DEXON available from United States Surgical Corporation, North Haven, Conn.], poly (L-lactide)(PLLA) [bone repair], poly (lactide/glycolide) [sutures sold under the tradenames VICRYL (90/10) and PANACRYL (95/5) available from Ethicon, Inc., Somerville, N.J.], poly (glycolide/epsilon-caprolactone (75/25) [sutures sold under the tradename MONOCRYL available from Ethicon, Inc., Somerville, N.J.], and poly (glycolide/trimethylene carbonate) [sutures sold under the tradename MAXON available from United States Surgical Corporation, North Haven, Conn.].

Other bulk erosion polymers are tyrosine derived poly amino acid [examples: poly (DTH carbonates), poly (arylates), and poly (imino-carbonates)], phosphorous containing polymers [examples: poly (phosphoesters) and poly (phosphazenes)], poly (ethylene glycol) [PEG] based block co-polymers [PEG-PLA, PEG-poly (propylene glycol), PEG-poly (butylene terephthalate)], poly (alpha-malic acid), poly (ester amide), and polyalkanoates [examples: poly (hydroxybutyrate (HB) and poly (hydroxyvalerate) (HV) co-polymers].

Of course, the polymer tubing may be made from combinations of surface and bulk erosion polymers in order to achieve desired physical properties and to control the degradation mechanism. For example, two or more polymers may be blended in order to achieve desired physical properties and stent degradation rate. Alternately, the polymer tubing may be made from a bulk erosion polymer that is coated with a surface erosion polymer.

In some embodiments, the polymeric tubing or stent provided may be comprised of blends of polymeric materials, blends of polymeric materials and plasticizers, blends of polymeric materials and therapeutic agents, blends of polymeric materials and radiopaque agents, blends of polymeric materials with both therapeutic and radiopaque agents, blends of polymeric materials with plasticizers and therapeutic agents, blends of polymeric materials with plasticizers and radiopaque agents, blends of polymeric materials with plasticizers, therapeutic agents and radiopaque agents, and/or any combination thereof. By blending materials with different properties, a resultant material may have the beneficial characteristics of each independent material. For example, stiff and brittle materials may be blended with soft and elastomeric materials to create a stiff and tough material. In addition, by blending either or both therapeutic agents and radiopaque agents together with the other materials, higher concentrations of these materials may be achieved as well as a more homogeneous dispersion. Various methods for producing these blends include solvent and melt processing techniques.

In one embodiment, plasticizers suitable for use in the present invention may be selected from a variety of materials including organic plasticizers and those like water that do not contain organic compounds. Organic plasticizers include but not limited to, phthalate derivatives such as dimethyl, diethyl and dibutyl phthalate; polyethylene glycols with molecular weights preferably from about 200 to 6,000, glycerol, glycols such as polypropylene, propylene, polyethylene and ethylene glycol; citrate esters such as tributyl, triethyl, triacetyl, acetyl triethyl, and acetyl tributyl citrates, surfactants such as sodium dodecyl sulfate and polyoxymethylene (20) sorbitan and polyoxyethylene (20) sorbitan monooleate, organic solvents such as 1,4-dioxane, chloroform, ethanol and isopropyl alcohol and their mixtures with other solvents such as acetone and ethyl acetate, organic acids such as acetic acid and lactic acids and their alkyl esters, bulk sweeteners such as sorbitol, mannitol, xylitol and lycasin, fats/oils such as vegetable oil, seed oil and castor oil, acetylated monoglyceride, triacetin, sucrose esters, or mixtures thereof. Preferred organic plasticizers include citrate esters; polyethylene glycols and dioxane.

In one embodiment, therapeutic agent or agents are combined with the polymeric intraluminal stent. Examples of therapeutic agents include but are not limited to: anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycin, plicamycin (mithramycin) and mitomycin, enzymes (L-asparaginase which systemically metabolizes L-asparagine and deprives cells which do not have the capacity to synthesize their own asparagines); antiplatelet agents such as G(GP) II_{b}/IIIₐ inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); anti-proliferative/antimitotic antimetabolites such as folic acid analogs (methotrexate), pyrimidine analogs (fluorouracil, floxuridine and cytarabine) purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory; such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetaminophen; indole and indene acetic acids (indomethacin, sulindac, and etodalec), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors, antisense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors.

The therapeutic agents may be incorporated into the stent in different ways. For example, the therapeutic agents may be coated onto the stent, after the stent has been formed, wherein the coating is comprised of polymeric materials into which therapeutic agents are incorporated. There are several conventional ways to coat the stents that are disclosed in the prior art. Some of the commonly used methods include spray coating; dip coating; electrostatic coating; fluidized bed coating; and, supercritical fluid coatings. Alternatively, the therapeutic agents may be incorporated into the polymeric materials comprising the stent. The therapeutic agent can be housed in reservoirs or wells in the stent design. The various techniques of incorporating therapeutic agents into, or onto, the stent may also be combined to optimize performance of the stent, and to help control the release of the therapeutic agents from the stent.

In another embodiment, radiopaque agents may be combined with the polymeric intraluminal stent. Because visualization of the stent as it is implanted in the patient is important to the medical practitioner for locating the stent, radiopaque agents may be added to the stent, which as described herein is a polymeric intraluminal stent. The radiopaque agents may be added directly to the polymeric materials comprising the stent during processing thereof resulting in fairly uniform incorporation of the radiopaque agents throughout the stent. The radiopaque agent can be housed in reservoirs or wells in the stent design. Alternately, the radiopaque agents may be added to the stent in the form of a layer, a coating, a band or powder at designated portions of the stent depending on the geometry of the stent and the process used to form the stent. Coatings may be applied to the stent in a variety of processes known in the art such as, for example, chemical vapor deposition (CVD), physical vapor deposition (PVD), electroplating, high-vacuum deposition process, microfusion, spray coating, dip coating, electrostatic coating, or other surface coating or modification techniques. Such coatings sometimes have less negative impact on the physical characteristics (i.e., size, weight, stiffness, flexibility) and performance of the stent than do other techniques. Preferably, the radiopaque material does not add significant stiffness to the stent so that the stent may readily traverse the anatomy within which it is deployed. The radiopaque material should be biocompatible with the tissue within which the stent is deployed. Such biocompatibility minimizes the likelihood of undesirable tissue reactions with the stent.

The radiopaque agents may include inorganic fillers, such as barium sulfate, bismuth subcarbonate, bismuth oxides and/or iodine compounds. The radiopaque agents may instead include metal powders such as tantalum, tungsten or gold, or metal alloys having gold, platinum, iridium, palladium, rhodium, a combination thereof, or other materials known in the art. Preferably, the radiopaque agents adhere well to the stent such that peeling or delamination of the radiopaque material from the stent is minimized, or ideally does not occur. Where the radiopaque agents are added to the stent as metal bands, the metal bands may be crimped at designated sections of the stent. Alternately, designated sections of the stent may be coated with a radiopaque metal powder, whereas other portions of the stent are free from the metal powder. The particle size of the radiopaque agents may range from nanometers to microns, preferably from less than or equal to 1 micron to about 5 microns, and the amount of radiopaque agents may range from 0-99 percent (wt. percent).

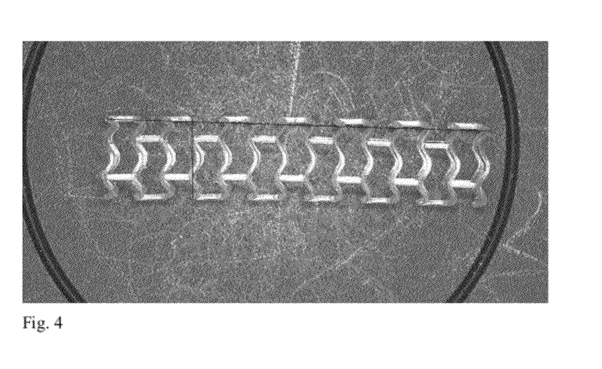

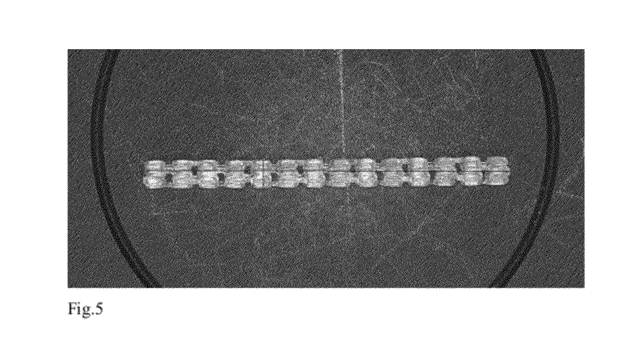

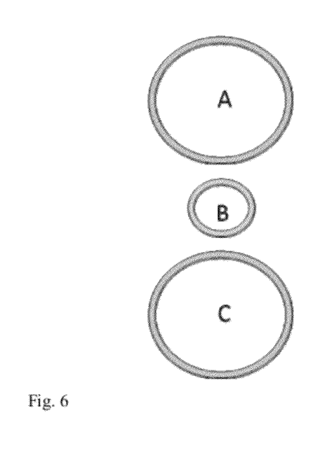

The novel process of the present invention starts with polymeric stents machined to a final desired size and configuration that would be representative of the stent after balloon deployment (as shown in FIG. 4 and FIG. 6A). The stent is then heated ideally to a sufficiently effective temperature between the glass transition temperature (Tg) and the melting temperature (Tm) of the material, most preferably to a temperature approximately 10° C.-20° C. above the Tg of the material. Heating may be achieved through various known means in the art, including heated water bath, environmental chamber, induction heating, and IR radiation, etc. Those skilled in the relevant art may recognize other means of heating that also fall within the scope of the present invention. The stent is held at this temperature for a sufficient predetermined amount of time (e.g., up to 30 seconds) to effectively ensure uniform heating of the stent, which is dependent on a number of factors, including the material, the amount of crystallinity, device thickness, as well as the part geometry. At this elevated temperature the stent is then subjected to a radial compression orientation process whereby the stent is radially compressed (as shown in FIG. 5 and FIG. 6B) to a certain prescribed smaller diametric size, over a sufficiently effective period of time (approximately 10 seconds), held at this temperature for a sufficiently effective period of time, i.e., about 30 seconds or less, and then cooled to substantially below the material's Tg while in this configuration. Radial compression can be achieved through any known process including, but not limited to, using a stent crimping apparatus, heat or cold shrink tubing, or elastic tubing, etc. Those skilled in the art may know other means of radial compression that can also be used within the scope of this invention. Since the stent is above the Tg of the material during the radial compression process the polymeric chains are oriented during the compression process as dictated by the stent geometry as it is being compressed. It may be desirable to radially compress the stent all the way to a final deliverable stent size on a balloon catheter, or to some interim diametral size (smaller than starting size) followed by crimping on the balloon catheter delivery apparatus at a temperature below the Tg of the material, typically 25° C.-50° C. as is typically done with crimping of stents. Such a size may be, but is not limited to an OD diameter range of 0.045″-0.080″; those skilled in the art will recognize other suitable interim sizes within the scope of the invention. After the device is radially compressed to the desired size it is cooled in this configuration. Cooling can be achieved through any known means including ice water, cool air or nitrogen, etc. The radial compression process being conducted at this elevated temperature (>Tg) effectively induces polymeric orientation in the stent struts while the stent is being crimped to a smaller diameter. Furthermore, the heating, radial compression orientation, cooling process can be achieved in one step or a series of multiple steps to sequentially smaller diameters which may enable more precise control the compression process.

During the radial compression orientation process stent struts are crimped to a smaller size and polymer orientation is induced in the regions of the stent geometry where strain and deformation occurs (see FIG. 5—photograph of stent following radial compression). These areas are dependent on stent geometry. A mandrel can be used on the stent ID to control device size and facilitate removal following radial compression. The post-orientation size of the stent is smaller than the starting size before radial compression and may be the desired insertion size of the stent. It is conceivable or perhaps desirable to radial compress the stent directly onto the delivery system (folded balloon) during this compressive orientation step. In lieu of this, there may be a separate crimping step to bring the final diameter down even further to the desired insertion size onto the balloon. This crimping step, if desired, may be facilitated by exposing the stent to a lower temperature than that used in the radial compression process, preferably a temperature below the glass transition temperature (Tg) of the material, which may be 40° C.-50° C. for PLA or PLGA based polymers. After insertion in the body as is known the stent art, the stent is deployed to desired size (as shown in FIG. 6C), typically via a balloon catheter at a pressure range of approximately 6-20 atm. Since the pre-orientation size of the stent is this deployed size (or even larger diameter) the stent will have a tendency to maintain (or even grow slightly larger) as known polymer material relaxation may occur in a beneficial direction of opposing stent recoil.

The following examples are illustrative of the principles and practice of the present invention, although not limited thereto.

### Example 1

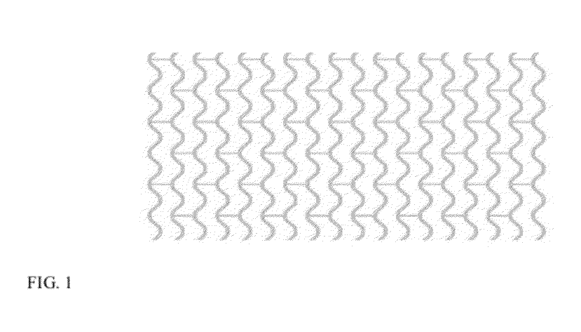

A stent having a configuration as seen in FIG. 1 was laser cut from a section of polymeric tubing with an outside diameter (OD) of 0.144″, inside diameter (ID) of 0.128″, and length of 17 mm, the desired final dimensions of the stent after balloon deployment. The tubing material was a blend of 90 wt. % 85/15 PLGA and 10 wt. % 35/65 PCL/PGA. A 2-D mask of the stent design was created and used to direct the excimer laser energy to ablate the desired, exposed regions of the tubing as it is rotated to form the stent. The laser-cut stent was placed in a stent crimper and heated to 70° C. (above the glass temperature (Tg) of the material) for less than 30 seconds, at which time the stent was then crimped under radial compression to an approximate OD of 0.080″ in about 10 seconds. The stent was held at this size for less than 30 seconds and then cooled in an ice bath (below the Tg of the material). The stent was then placed on a 3.0 mm balloon catheter and heated in a water bath at 37 C for 1 minute. After 1 minute of preheating the stent was a pressure of 12 atm. was applied and held for 1 minute. Following balloon deployment the stents submerged in a 37° C. to measure the stent recoil over time with the following results (est. measurement error +/−1%) as seen in Table 1.

TABLE 1

| Hours in water | |
|---|---|
| bath | Recoil |
| 24 | 1.7% |
| 74 | 2.2% |
| 120 | 2.3% |
| 145 | 1.2% |
| 239 | 1.0% |
| 287 | 0.0% |

### Example 2

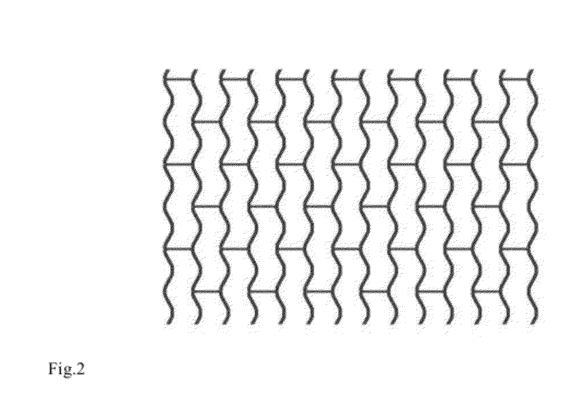

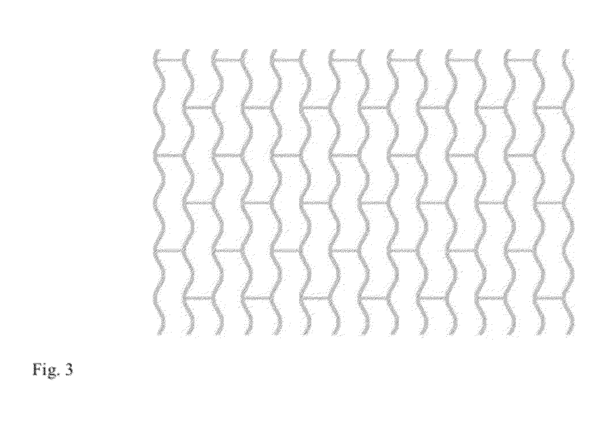

Stents having configurations as seen in FIG. 2 and FIG. 3 were laser cut from polymeric tubing (90 wt. % 85/15 PLGA and 10 wt. % 35/65 PCL/PGA) with an outside diameter (OD) of 0.144″, inside diameter (ID) of 0.128″, and length of 17 mm, the desired final dimensions of the stents after balloon deployment. The laser cut stents were individually placed in a stent crimper and heated to 70° C. (above the glass temperature (Tg) of the material) for less than 30 seconds, at which time the stents were then oriented and crimped under radial compression to an approximate OD of 0.057″ in about 10 seconds (see FIG. 5). The stents were held at this size for less than 30 seconds and then cooled in an ice bath (below the Tg of the material). Stents were placed on a 3.0 mm balloon catheter and heated in a water bath at 37° C. for 1 minute. After 1 minute of preheating the stent 16 atm of pressure was applied and held for 1 minute. Following balloon deployment (see FIG. 4) the stents submerged in a 37° C. to measure the stent recoil over time with the following results (estimated measurement error +/−1%) as presented in Table 2.

TABLE 2

| | FIG. 2 | FIG. 5 |
|---|---|---|
| Hours in | Design | Design |
| Water Bath | Recoil | Recoil |
| 27 | −0.5% | 1.3% |
| 96 | −0.5% | 0.0% |
| 147 | −2.3% | −0.8% |
| 192 | −2.5% | 0.0% |
| 288 | −2.8% | −0.8% |

Negative recoil indicates sizes that are larger than the maximum size as balloon inflation which was typically within 2-3% of the original starting diameter of the tubing (pre radial compression orientation size). As can be seen instead of typical polymer stents made from other methods which tend to shrink in diameter leading to stent recoil, stents made from this method have a driving force to return to their original size. Depending on the balloon pressure and size of deployment, the stents have the potential to actually have negative recoil (grow in size larger than their deployed size).

### Example 3

Two stents with a configuration shown in FIG. 3 were laser cut from polymeric tubing (90 wt. % 85/15 PLGA and 10 .wt. % 35/65 PCL/PGA) with an outside diameter (OD) of 0.144″, inside diameter (ID) of 0.128″, and length of 17 mm. Stent A was individually placed in a stent crimper and heated to 70° C. (above the glass temperature (Tg) of the material) for less than 30 seconds, at which time the stents were then oriented and crimped under radial compression to an approximate OD of 0.057″ in about 10 seconds. The stent was held at this size for less than 30 seconds and then cooled in an ice bath (below the Tg of the material). Stent A was placed on a 3.0 mm balloon catheter and heated in a water bath at 37° C. for 1 minute. After 1 minute of preheating the stent was inflated at a pressure of 16 atm was applied and held for 1 minute. Following balloon deployment stent A was submerged in a 37° C. to measure the stent recoil over time with the following results (estimated measurement error +/−1%) as presented in Table 2. Stent B was processed in an identical manner except prior to laser cutting the tubing was “educated” at 80° C. for 30 minutes as described in Lafont et al. (U.S. Pat. No. 7,731,740) (estimated measurement error +/−1%) as presented in Table 3.

TABLE 3

| Hours in | | |
|---|---|---|
| Water | Stent A Design | Stent B Design |
| Bath | Recoil | Recoil |
| 0.1 | 0.1% | 1.3% |
| 3 | 5.7% | 4.0% |
| 6 | 4.5% | 4.6% |
| 72 | 2.2% | 2.6% |
| 98 | 2.3% | 3.1% |
| 121 | 0.4% | 2.5% |
| 170 | −1.3% | 2.1% |
| 247 | −1.9% | 1.1% |
| 290 | −2.8% | 0.7% |

As can be seen both stent A and stent B produced a polymeric based stent that initially exhibited some initial acute recoil that was later resolved over time. Stent B was processed identical to stent A after the tubing to construct stent B was “educated” by the example thermal process as described by Lafont et al. Stent A was not educated and yet it recovered its initial acute recoil fully and at a faster rate than stent B that was “educated” as per Lafont et al.

### Example 4

A stent with a configuration shown in FIG. 3 was laser cut from polymeric tubing (90 wt. % 85/15 PLGA and 10 wt. % 35/65 PCL/PGA) with an outside diameter (OD) of 0.144″, inside diameter (ID) of 0.128″, and length of 17 mm. The stent was placed in a stent crimper and heated to 70° C. (above the glass temperature (Tg) of the material) for less than 30 seconds, at which time the stents were then oriented and crimped under radial compression to an approximate OD of 0.057″ in about 10 seconds. The stent was held at this size for less than 30 seconds and then cooled in an ice bath (below the Tg of the material). The stent was placed on a 3.0 mm balloon catheter and heated in a water bath at 37° C. for 1 minute. After 1 minute of preheating the stent was inflated to a low pressure of 4 atm and held for 1 minute. Following balloon deployment the stent was submerged in a 37° C. to measure the stent recoil over time with the following results (estimated measurement error +/−1%) as presented in Table 4 (estimated measurement error +/−1%).

TABLE 4

| Hours in | Stent Recoil (after |
|---|---|
| Water Bath | 4 atm. deployment |
| 0.1 | 7.3% |
| 3 | 10.4% |
| 6 | 7.3% |
| 72 | 4.9% |
| 98 | 4.3% |
| 121 | 5.9% |
| 170 | 5.2% |
| 247 | 5.0% |
| 290 | 4.8% |
| 365 | 1.1% |

In this example, stents processed according to this description were deployed at an extremely low balloon pressure of 4 atm. to demonstrate ability to recover from stent recoil. Initial acute recoil was high at about 10% at 3 hours post-deployment, likely due to the low level of plastic deformation imparted to the stent, but gradually the recoil reduced over time as the stent grew in size, resulting in an almost complete recovery by 15 days within the measurement error.

The method of manufacturing intraluminal stents described herein produces polymeric stents having reduced recoil. The diametral size that a stent is manufactured to (prior to radial compression orientation) is the equilibrium diametral size programmed into the stent and the size it will seek over time as the stent relaxes from its temporary polymeric orientation state. The use of balloon deployment accelerates the process and provides consistency of stent delivery with currently known methods such that the stent relaxation from the oriented, crimped condition serves as the driving force to inhibit stent recoil over time. The relaxation serves to grow the stent diameter as opposed to other methods in the art which must deal with driving forces and polymeric material creep and relaxation that would cause a decrease in stent diameter and higher stent recoil over time. A further advantage of the disclosed method is that it does not depend upon the specific stent design utilized and those skilled in the art will soon recognize that the process is applicable in a similar manner to various stent designs known in the art and equivalents. Stents manufactured by such a process can be inserted in the body in a crimped/oriented configuration and deployed with a balloon catheter or other equivalent device. The balloon expansion step takes the stent directly to the final desired diameter. It is recognized that polymeric materials may relax or creep at significantly different rates back to their equilibrium state and it is this very behavior that will serve as the driving force to limit stent recoil. Generally speaking, stents made from more amorphous and/or elastic polymers may achieve this relaxation effect to final desired size more quickly than brittle or highly crystallized materials. Amorphous materials may be desirable in the body since they tend to not contain crystalline regions that may be more immunogenic in the body.

The polymer tubing that is provided may be prepared by conventional methods such as extrusion, injection molding, and solvent casting. The desired polymer tubing diameter and wall thickness are dependent on the final diameter of the stent, which is in turn dependent on the diameter of the body lumen in which the stent will be deployed. One of skill in the art will be able to determine the appropriate polymer tubing diameter and wall thickness with the benefit of the invention described herein.

Polymers have two thermal transitions; namely, the crystal-liquid transition (i.e., melting point temperature, Tₘ) and the glass-liquid transition (i.e., glass transition temperature, T_{g}). In the temperature range between these two transitions there may be a mixture of orderly arranged crystals and chaotic amorphous polymer domains. The glass transition temperature, Tg, is the temperature at atmospheric pressure at which the amorphous domains of a polymer change from a brittle vitreous state to a solid deformable or ductile state. At temperatures above the Tg segmental motion of the polymer chains occur. It is desirable to maintain high strength and limit creep or recoil of the stents disclosed herein for proper function. For this purpose it is desirable to use polymers with a Tg greater than body temperature.

Molecular orientation of the polymer chains can be obtained in the following manner: The polymer stent having diameter A is placed in the radial compression device, such as a stent crimper and heated above the T_{g} of the polymer, preferably about 10-20° C. above the T_{g} for a certain period of time. Any known means of heating may be used including but not limited to a heated water bath, heated inert gas, such as nitrogen, and heated air. It is desirable to heat the polymeric stent uniformly and the time required depends on the thickness, surface area and mode of heating applied. For thin polymer stents (150-200 microns) the heating time may be approximately 20 seconds to 1 minute prior to radial compression. The radial compression may be performed while the stent is placed on a mandrel. The compressed stent is then quickly cooled to below the Tg of the polymer through any known means (ice bath, cooled nitrogen or air, etc.).

The above descriptions are merely illustrative and should not be construed to capture all consideration in decisions regarding the optimization of the design and material orientation. It is important to note that although specific configurations are illustrated and described, the principles described are equally applicable to many already known stent configurations. Although shown and described is what is believed to be the most practical and preferred embodiments, it is apparent that departures from specific designs and methods described and shown will suggest themselves to those skilled in the art and may be used without departing from the spirit and scope of the invention. The present invention is not restricted to the particular constructions described and illustrated, but should be constructed to cohere with all modifications that may fall within the scope for the appended claims.

What is claimed is:

1. A method of manufacturing a polymeric stent, comprising the steps of:
forming a polymeric stent from a polymeric material, the stent having a first inner diameter and a first outer diameter, such that the stent has a plurality of openings forming struts, wherein the first inner and first outer diameters of the stent are substantially equal to the inner and outer diameters of the stent post-deployment;
heating the stent to a temperature sufficiently above the Tg of the material;
radially compressing the stent at the temperature such that it has a reduced second inner diameter and a reduced second outer diameter, wherein the second inner and outer diameters are smaller than the first inner and outer diameters, respectively; and,
cooling the stent in the compressed configuration,
wherein the stent has substantially no recoil after deployment.

2. The method of claim 2, wherein the polymeric material comprises a poly (α-hydroxy ester) polymer selected from the group consisting of, poly (lactic acid), poly (glycolic acid), poly (caprolactone), poly (p-dioxanone), poly (trimethylene carbonate), poly (oxaesters), poly (oxaamides), and copolymers and blends thereof.

3. The method of claim 1, wherein the stent additionally comprises a therapeutic agent.

4. The method of claim 3, wherein the therapeutic agent is selected from the group consisting of anti-restenotic agents, anti-thrombotic agents, anti-proliferative/antimitotic agents, anti-coagulant, anti-inflammatory, and immunosuppressive agents.

5. The method of claim 1, wherein the temperature is about 5° C. to about 20° C. above the Tg.

6. The method of claim 1, wherein in the stent is formed by laser cutting a polymeric tube.

7. A polymeric stent manufactured by a process comprising the steps of:
forming a polymeric stent from a polymeric material, the stent having a first inner diameter and a first outer diameter, such that the stent has a plurality of openings forming struts, wherein the first inner and first outer diameter of the stent are substantially equal to the inner and outer diameters of the stent post-deployment;
heating the stent to a temperature sufficiently above the Tg of the material;
radially compressing the stent at the temperature such that it has a reduced second inner diameter and a reduced second outer diameter, wherein the second inner and outer diameters are smaller than the first inner and outer diameters, respectively; and,
cooling the stent in the compressed configuration,
wherein, the stent, when expanded to a size substantially equal to the first inner diameter and the first outer diameter, has substantially no recoil.

8. The stent of claim 7, wherein the polymeric material comprises a poly (α-hydroxy ester) polymer selected from the group consisting of, poly (lactic acid), poly (glycolic acid), poly (caprolactone), poly (p-dioxanone), poly (trimethylene carbonate), poly (oxaesters), poly (oxaamides), and copolymers and blends thereof.

9. The stent of claim 7, wherein the stent additionally comprises a therapeutic agent.

10. The stent of claim 9, wherein the therapeutic agent is selected from the group consisting of anti-restenotic agents, anti-thrombotic agents, anti-proliferative/antimitotic agents, anti-coagulant, anti-inflammatory, and immunosuppressive agents.

11. The stent of claim 7, wherein the temperature is about 5° C. to about 20° C. above the Tg.

12. The stent of claim 7, wherein in the stent is formed by laser cutting a polymeric tube.
